(19) United States
(12) Patent Application Publication
(10) Pub. No.: US 2022/0287567 A1
(43) Pub. Date: Sep. 15, 2022

(54) SYSTEM AND METHOD FOR MEASURING TISSUE PARAMETERS BY USE OF CAPACITIVE TACTILE SENSOR

(71) Applicant: UE LifeSciences Inc., Philadelphia, PA (US)

(72) Inventors: Matthew S. CAMPISI, Commack, NY (US); Mihir SHAH, Chester Spring, PA (US); Bhaumik SANGHVI, Mumbai (IN)

(21) Appl. No.: 17/627,612

(22) PCT Filed: Jul. 16, 2020
(86) PCT No.: PCT/US2020/042298

§ 371 Date: Jan. 14, 2022

Related U.S. Application Data
(60) Provisional application No. 62/875,485, filed on Jul. 17, 2019.

(51) Int. Cl. A61B 5/00

(52) U.S. Cl. CPC A61B 5/0053; A61B 5/4312; A61B 5/442; A61B 5/7275; A61B 5/741; A61B 5/7445; A61B 2562/0214

(57) ABSTRACT

A device and method for detecting, documenting, measuring and mapping the size, shape and location of lesions underlying the surface of the skin or other soft tissue in a subject by measuring variations in the tactile pressure of the skin or other tissue surface being investigated through the use of a capacitive tactile sensor is described. A capacitive tactile sensor comprises at least one exposure window, a substrate comprising a plurality of electrodes disposed within the housing, an insulating layer, a non-conductive compressible membrane and covering positioned over the insulating layer wherein at least a portion of the membrane is accessible via the exposure window, and a controller configured to calculate at least one parameter of a tissue of a subject. A method of measuring a tissue parameter in a subject and a method of performing a self-guided examination on a tissue of a subject are also described.

### CROSS-REFERENCE TO RELATED APPLICATIONS

This application claims priority to U.S. Provisional Patent Application No. 62/875,485, filed on Jul. 17, 2019, incorporated herein by reference in its entirety.

### BACKGROUND OF THE INVENTION

The key to successful treatment of many forms of cancer lies in early detection. In turn, the early detection and identification of cancerous growths is heavily dependent upon the availability, relative costs, effectiveness and associated risks of existing sensor and screening technologies. Currently, there are a variety of different sensors and tools used for investigating the mechanical properties of soft tissue and for imaging soft tissue.

One type of conventional soft tissue sensor uses an external force applicator for inducing displacement and an external displacement gauge for measuring resistive force. The external force applicator may be hydraulic or piezoelectric, and the external displacement gauge may be optical or piezoelectric.

Exemplary soft tissue imaging tools include Computer Tomography (CT), Magnetic Resonance Imaging (MRI), Ultrasound (US), T-scan (TS) and Ultrasound elastography (UE). CT scans take 360-degree X-ray pictures and reconstruct 3D tissue structures using computer software. MRI scans use powerful magnetic fields and radio waves to create tissue images for diagnosis. US scans transmit high frequency waves through tissue and capture the echoes to image tissue structures. TS measures low-level bioelectric currents to produce real-time images of electrical impedance properties of tissues. UE scans evaluate the echo time through tissue under a constant mechanical stress and compares it to that of the same tissue when unstressed. A tissue strain map is then obtained, from which an image of 2D elastic modulus distribution is created by conventional inversion techniques.

Tactile imaging tools, such as mammography, use array pressure sensors to probe spatial tissue stiffness variations. Currently, mammography is used in breast cancer screening to detect abnormal tissue by tissue density contrast. Mammography is the only FDA approved breast cancer screening technique, which has a typical sensitivity of 85% that decreases to 65% in radio-dense breasts. However, in these screening processes there is a high incidence of false positives. In fact, only about 15-30% of breast biopsies yield a diagnosis of malignancy. Although effective for screening women over 40, mammography is not as effective for screening women who have dense breast tissue.

Since many tissues harboring abnormal growths are stiffer than the surrounding normal tissues under compression, detecting a change in tissue stiffness has increasingly become an important factor in the detection of potentially abnormal tissue. For example, breast cancers are calcified tissues that are known to be more than seven times stiffer than normal breast tissue. Similarly, plaque-lined blood vessels are also stiffer than normal, healthy blood vessels.

U.S. Pat. No. 7,497,133, incorporated herein by reference, discloses a piezoelectric finger sensor that may be used to detect tumors by measuring tissue stiffness. Tumor mobility was assessed from the ratio of the shear modulus to the elastic modulus (G/E) ratio of the tumor or by sensitive direct tumor mobility measurement using two piezoelectric finger sensors, one for pushing and one for measuring the movement of the tumor that results from the pushing. The patent concludes that the G/E ratio is higher in a tumor region than the G/E ratio for surrounding normal tissue and that a much higher G/E ratio in the cancer region indicated that the tumor was less mobile under shear than under compression, as compared to the surrounding normal tissue. Although the patent concludes that these measurements may offer the potential for non-invasive breast cancer malignancy screening, it does not disclose a method for determining malignancy, invasiveness, or tumor type.

U.S. Pat. No. 8,562,546, incorporated herein by reference, discloses a piezoelectric sensor system for evaluating tissue, including determination of whether the tissue contains abnormal growths. The described system uses an array of piezoelectric elements which are actuated in a first direction toward the tissue, after which their relative positions are recorded in order to approximate tissue firmness. Such sensors are very difficult to calibrate and can fall out of calibration after continuous use, or even accidental touching. They are therefore not suitable for use in the hands of a layperson.

Consequently, there remains an important need for an accurate, non-invasive, inexpensive, portable, and easy-to-use system to detect, document, measure and map the size, shape and location of lesions underlying the surface of the skin or other soft tissue. The present invention satisfies this need.

### SUMMARY OF THE INVENTION

In one embodiment, a device for measuring a tissue parameter in a subject includes a housing having at least one exposure window; a substrate comprising a plurality of electrodes disposed within the housing; an insulating layer positioned over the electrodes; a pliable membrane positioned over the insulating layer wherein at least a portion of the pliable membrane is accessible via the exposure window; and a controller disposed within the housing configured to calculate at least one parameter of a tissue of a subject based on a capacitance measured between the electrodes. In one embodiment, the pliable membrane comprises a foam. In one embodiment, the foam has a hardness in a range of 00-0 to 00-20. In one embodiment, the plurality of electrodes comprises at least 2 electrodes. In one embodiment, each of the plurality of electrodes has a surface area of between 1 mm² and 16 mm². In one embodiment, the device includes a visualization device communicatively connected to the controller, the visualization device comprising a display. In one embodiment, the controller is connected to the visualization device via a Bluetooth connection. In one embodiment, the device includes a conducting element configured to deliver a voltage reference to a surface in contact with the pliable membrane. In one embodiment, the conducting element is positioned substantially surrounding the exposure window. In one embodiment, the at least one parameter comprises tissue firmness. In one embodiment, the tissue of the subject comprises breast tissue.

In one embodiment, a method of measuring a tissue parameter in a subject includes the steps of positioning a handheld device comprising a plurality of electrodes and a quantity of pliable material substantially in contact with a body region of a subject; measuring a capacitance between two electrodes of the plurality of electrodes; determining, based on the capacitance, the thickness of the pliable material; and calculating a tissue parameter based on the thickness of the pliable material. In one embodiment, the method includes transmitting data selected from the group consisting of the capacitance, the thickness, and the tissue parameter to a visualization system. In one embodiment, the method includes measuring a plurality of capacitance values between a set of electrode pairs, and displaying on the visualization system a diagram of parameter values across the tissue surface. In one embodiment, the tissue parameter is firmness. In one embodiment, the method includes the step of making a provisional diagnosis based on the tissue parameter.

In one embodiment, a method of performing a guided self-examination on a tissue of a subject includes the steps of positioning a handheld device comprising a plurality of electrodes and a quantity of pliable material substantially in contact with a body region of a subject; measuring a capacitance between two electrodes of the plurality of electrodes; calculating a tissue parameter based on the capacitance; determining, in a controller, an instruction for the subject based on the tissue parameter; and issuing the instruction to the subject in order to induce the subject to manipulate the handheld device. In one embodiment, the instruction is an auditory instruction issued via a speaker. In one embodiment, the instruction is configured to induce the subject to move the device to a new location on the tissue. In one embodiment, the instruction comprises a visual cue, presented on a display.

In one embodiment, a capacitive tactile sensor device for detecting, documenting, measuring and mapping the size, shape and location of lesions underlying the surface of the skin or other soft tissue in a subject by measuring variations in the tactile pressure of the skin or other tissue surface, includes a housing having at least one exposure window and exposed conductive surfaces placed so that the operator or the device will be in contact with the conductive surfaces at all times while operating the device; a substrate located within the housing measurements comprising a plurality of pairs of separate coplanar, co-located electrodes, arranged in in a Cartesian grid, on an electrically non-conductive material, and configured such that any two adjacent electrodes are electrically independent and can be stimulated independently by an externally generated stimulating voltage or current signal, creating a capacitor between each such electrode pair, with the space above the outward surface of the electrode grid serving as a dielectric; a layer of insulating material positioned over the outward facing surface of the electrode grid; a homogeneous, compressible, non-ferrite and non-conductive membrane covering the insulating layer positioned over the outward facing surface of the electrode grid, which membrane: (a) is of uniform thickness across the entire surface area of each pair of adjacent electrodes forming a capacitor, and across the entire surface area of the Cartesian grid of electrodes; (b) has a fixed, uniform and known compression ratio and hardness across the entire surface area of the Cartesian grid of electrodes; and (c) has a steady-state equilibrium shape such that, when not subject to external forces, its volume remains constant; and a non-conductive cover material positioned over the outward facing surface of the homogeneous, compressible, non-ferrite and compressible membrane, at least a portion of which is accessible through the exposure window of the housing, which serves as the outward facing surface of the sensor and which, together with the membrane, effectively replaces air as a dielectric for the capacitor created by each pair of adjacent electrodes, one functioning as the transmitting electrode and one functioning as the receiving electrode; wherein the sensor surface is configured to be placed completely and firmly in contact with the surface of the tissue of the subject such that the electromechanical properties including normal surface pressure of the conductive tissue will disrupt the electric field in the dielectric comprised of the compressible membrane and disturb the capacitive coupling between the relevant pairs of coplanar electrodes; wherein the sensor input device induces and generates at least one signal in response to the pressure imposed upon the sensor surface by contact with the skin or other soft tissue surface, and the resulting compression of the compressible membrane and disturbance of the capacitive coupling between the relevant pairs of coplanar electrodes, in accordance with the varying composition and properties of the underlying tissue structure, as an indicator of the location, dynamic and special features of localized areas of stiffer tissue underlying the area of skin or other tissue surface being investigated; and a controller disposed within the housing comprised of: (a) one or more commercially available IC capacitive sensor chips configured: (i) to generate signals used to stimulate the electrode pairs located in the substrate; (ii) to receive and process the signals subsequently induced, generated and transmitted by the capacitive tactile sensor input device in response to the pressure imposed upon the sensor surface by contact with the skin or other soft tissue surface; and (iii) to induce, generate and transmit to the device signals derived from the processing of the signals induced, generated and transmitted to the IC capacitive sensor chip(s) by the capacitive tactile sensor input device; and (b) one or more integrated circuits and processors configured (i) to receive and process the signals induced, generated and transmitted by the IC capacitive sensor chip(s) to calculate one or more parameters of the underlying tissue structure, based on changes in the perceived capacitance between the adjacent relevant electron pairs caused by the pressure imposed upon the sensor surface by contact with the skin or other soft tissue surface; and (ii) to communicatively connect with a visualization device comprising a display and an integrated storage device. In one embodiment, the homogeneous, compressible, non-ferrite and non-conductive membrane comprises a polyurethane, silicon, or thermoplastic elastomer foam. In one embodiment, the foam has a hardness in a range of 00-0 to 00-20. In one embodiment, the plurality of electrodes comprises at least 2 electrodes. In one embodiment, each of the plurality of electrodes has a surface area of between 1 mm² and 16 mm². In one embodiment, the device includes a visualization device communicatively connected to the controller, the visualization device comprising a display. In one embodiment, the controller is connected to the visualization device via a Bluetooth connection. In one embodiment, includes conducting elements on the exterior of the housing configured to deliver a voltage reference used for all subsequent capacitive calculations. In one embodiment, the device includes a grounding pad or strap providing conductive contact between an uninsulated portion of the electron plane and the external surface of the capacitive sensor, configured to deliver a voltage reference used for all subsequent capacitive calculations. In one embodiment, the at least one parameter comprises tissue firmness. In one embodiment, the tissue of the subject comprises breast tissue.

In one embodiment, a method of documenting, measuring and mapping the size, shape and location of palpable lesions underlying the surface of the skin or other soft tissue in a subject by measuring variations in the normal tactile pressure on the surface of the skin or other soft tissue areas of the tissue surface using capacitive tactile sensing includes the steps of positioning a handheld device comprising a plurality of electrodes and a quantity of non-conductive compressible material and covering substantially in contact with the surface area of the skin or tissue of a subject; determining a baseline reference capacitance of the subject by measurements obtained by the operator from a lesion-free area of tissue; determining a baseline electrical voltage potential reference from the operator through conductive contact between the operator and the handheld device; positioning the handheld device substantially in contact with the surface area of the skin or tissue being investigated; measuring capacitance between two electrodes of the plurality of electrodes resulting from contact with the surface area of the skin or tissue being investigated; measuring variations in the perceived capacitance between the adjacent relevant electrode pairs, with reference to the baseline electrical voltage potential references, caused by the pressure imposed upon the sensor surface (compressible membrane and covering), resulting from the disruption to the capacitive coupling between the adjacent relevant electrode pairs, caused by contact with the skin or surface of the tissue being investigated; and calculating one or more tissue parameters based on the variations in perceived capacitance from the baseline electrical voltage potential references. In one embodiment, the method includes the step of transmitting data selected from the group consisting of the perceived capacitance, the thickness, and the tissue parameter to a visualization system. In one embodiment, the method includes the step of measuring a plurality of capacitance values between a set of electrode pairs and displaying on the visualization system a diagram of parameter values across the tissue surface. In one embodiment, the tissue parameter is firmness.

In one embodiment, a method of performing a guided self-examination on a tissue of a subject includes the steps of positioning a handheld device comprising a plurality of electrodes and a quantity of pliable material substantially in contact with a body region of a subject; measuring a capacitance between two electrodes of the plurality of electrodes; calculating a tissue parameter based on the capacitance; determining, in a controller, an instruction for the subject based on the tissue parameter; and issuing the instruction to the subject in order to induce the subject to manipulate the handheld device. In one embodiment, the instruction is an auditory instruction issued via a speaker. In one embodiment, the instruction is configured to induce the subject to move the device to a new location on the tissue. In one embodiment, the instruction comprises a visual cue, presented on a display.

In one aspect, a method of dynamically calibrating data received from at least one sensor comprises acquiring a plurality of data values from at least one sensor, calculating a first mean and a first standard deviation of the plurality of data values, obtaining a subset of the plurality of data values, whose values are within one standard deviation of the first mean, calculating a second mean from the subset of the plurality of data values as a baseline value, and subtracting the baseline value from the plurality of data values to generate a calibrated data set.

In one embodiment, the method further comprises acquiring a second plurality of data values, calculating a third mean and third standard deviation of the first and second pluralities of data values, obtaining a second subset of the first and second pluralities of data values, whose values are within one standard deviation of the third mean, calculating a fourth mean from the second subset of the pluralities of data values as a second baseline value, and subtracting the second baseline value from the first and second pluralities of data values to generate the calibrated data set.

In one embodiment, the method further comprises displaying a three-dimensional surface plot of the calibrated data set, displaying baseline value, and providing visual feedback when the baseline value is within an optimal range. In one embodiment, the visual feedback comprises changing a color of a region of the three-dimensional surface plot. In one embodiment, the method further comprises calculating a moving average of the plurality of data values and calculating the first mean and first standard deviation from the moving average.

In one embodiment, the method further comprises comparing the moving average to a maximum and a minimum threshold, capturing the plurality of data values or the moving average when the moving average is between the maximum and minimum thresholds, and storing the captured data values on a non-transitory computer-readable medium when the moving average remains between the maximum and minimum thresholds for a specified period of time. In one embodiment, the specified period of time is at least three seconds.

### BRIEF DESCRIPTION OF THE DRAWINGS

The foregoing purposes and features, as well as other purposes and features, will become apparent with reference to the description and accompanying Figures below, which are included to provide an understanding of the invention and constitute a part of the specification, in which like numerals represent like elements, and in which:

FIG. 1 is a diagram of a capacitive sheet according to one embodiment;

FIG. 2 is a diagram of a capacitive sheet according to one embodiment;

FIG. 3A is a cross section of an electrode pair on a substrate according to one embodiment;

FIG. 3B is a cross section of part of a capacitive tactile sensor device according to one embodiment;

FIG. 3C is an exemplary illustration of the normal pressure of the surface of tissue being applied to part of the capacitive tactile sensor device according to one embodiment;

FIG. 3D is an exemplary cross section of part of a capacitive tactile sensor input device in contact with an uneven surface according to one embodiment;

FIG. 4A and FIG. 4B are first and second embodiments of a housing having operator grounding contacts;

FIG. 5 is a visualization display graphic according to one embodiment;

FIG. 6A, FIG. 6B, and FIG. 6C are visualization display graphics according to one embodiment;

FIG. 7 a method of the present invention according to one embodiment; and

FIG. 8 is a chart of sample comparison data for piezo vs. capacitive sensor according to experimental examples.

### DETAILED DESCRIPTION

It is to be understood that the Figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, Python, PHP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the term “diagnosis” refers to the determination of the presence of a disease or disorder. In some embodiments of the present invention, methods for making a diagnosis are provided which permit determination of the presence of a particular disease or disorder.

As used herein, the term “screening” refers to the detection, documentation, measurement and/or mapping of the size, shape and location of abnormal features of the tissue of a subject which indicate the potential presence of disease or disorder warranting further investigation.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

One aspect of the present invention relates to a device for detecting characteristics of a tissue surface, including normal surface pressure and variations in such surface pressure over areas being investigated, which may indicate the presence of underlying tissue abnormalities, for example lumps, lesions, cysts, or tumors. An exemplary device of the present invention may be handheld or in a compact form factor. In some embodiments, a device of the present invention comprises multiple parts, while in other embodiments, the device is a self-contained and powered diagnostic device.

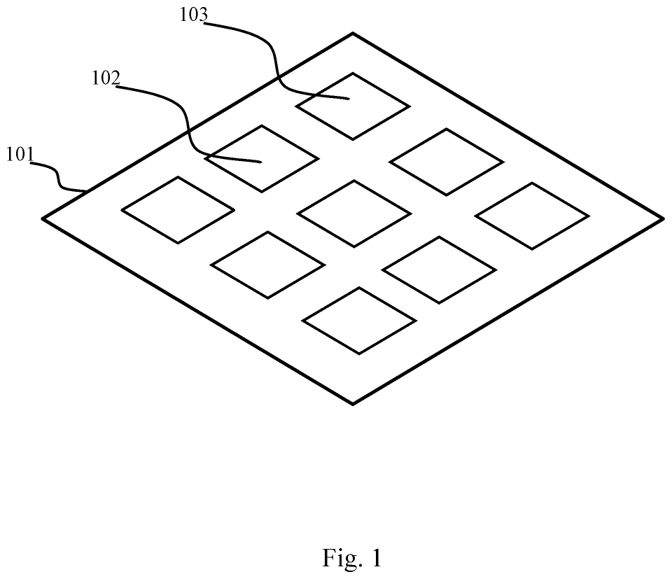

A device of the present invention may include a capacitive sheet as shown in FIG. 1. Capacitive sheet 101 may be rigid, flexible, or conformable, may be made of any suitable material, for example Kapton, glass fiber reinforced (fiberglass) epoxy resin, or reinforced phenolic resin. The depicted exemplary capacitive sheet includes a plurality of capacitive elements 102, 103. The depicted sheet includes nine capacitive elements or electrodes in a 3×3 Cartesian grid arrangement, but it is understood that capacitive sheets of the present invention may include any number of individual capacitive elements, for example in a range from 2 to a matrix of more than 20 by 20.

The depicted capacitive elements are squares, but it is understood that capacitive elements could be any shape, including but not limited to circles, triangles, hexagons, or squares. Individual capacitive elements may be made from any conducting material, including but not limited to copper, gold, silver, steel, aluminum, carbon nanotubes, stainless steel, or platinum.

Capacitive elements in a capacitive sheet may be any size, for example in a range from 1 mm² to 16 mm², and may have a thickness in a range of 0.5 mils to 3 mils typically. In some embodiments, all capacitive elements in a capacitive sheet are substantially the same size, but in other embodiments a capacitive sheet of the present invention may include a plurality of capacitive elements having different sizes. Similarly, different capacitive elements on the same sheet may be made of the same or different materials, or be the same or a different shape.

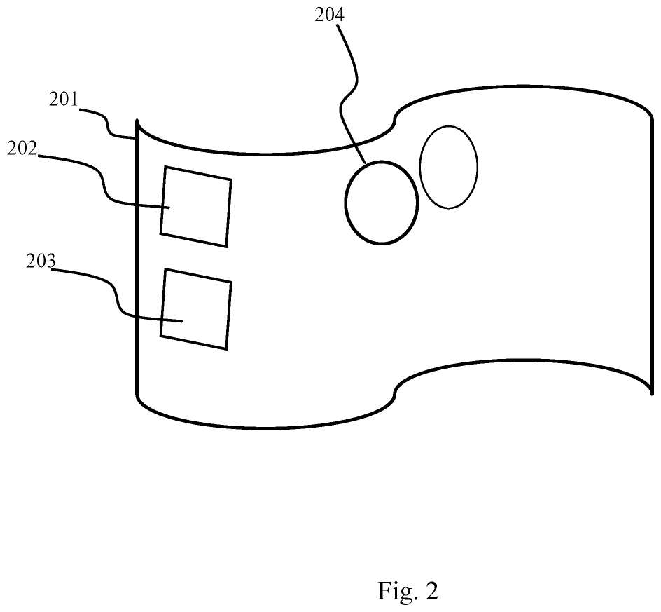

With reference now to FIG. 2, an alternative capacitive sheet 201 is shown. Exemplary sheet 201 includes substantially square capacitive elements 202 and 203, as well as substantially circular capacitive elements, for example 204.

The individual capacitive elements may in some embodiments be interconnected, and may be arranged such that any two adjacent electrodes are electrically independent and can be excited independently. In such a configuration, each pair of electrodes may be used as a separate capacitor, with the space above the surface of the capacitive sheet (including any materials placed or deposited thereon) acting as the dielectric.

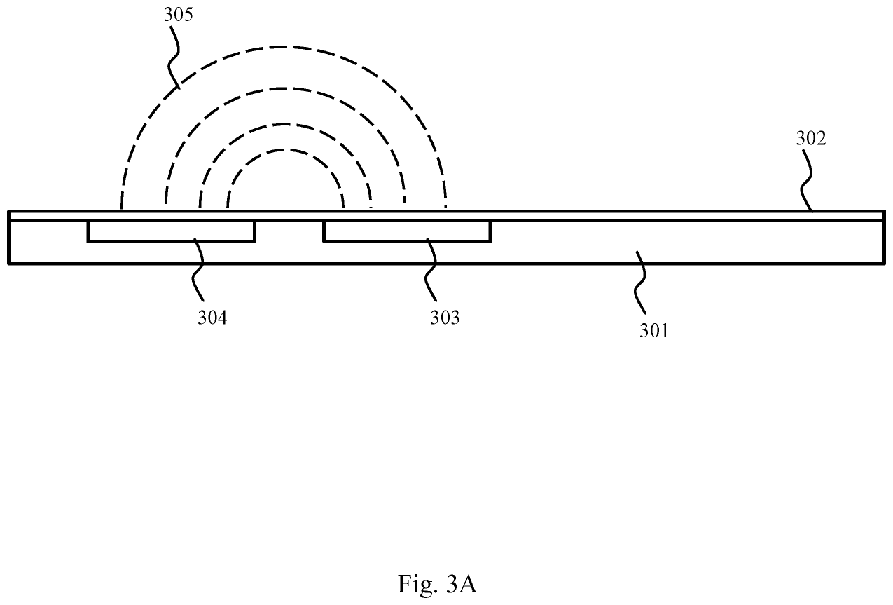

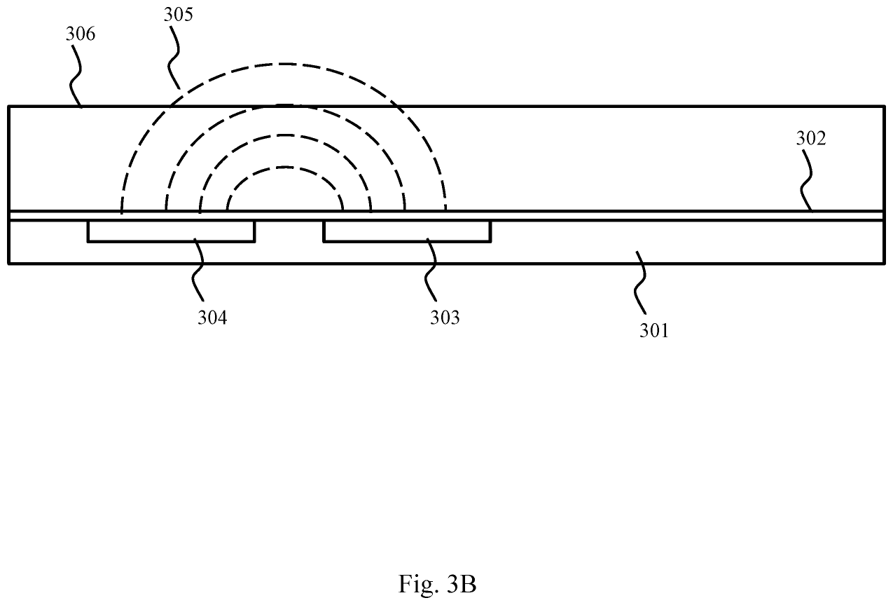

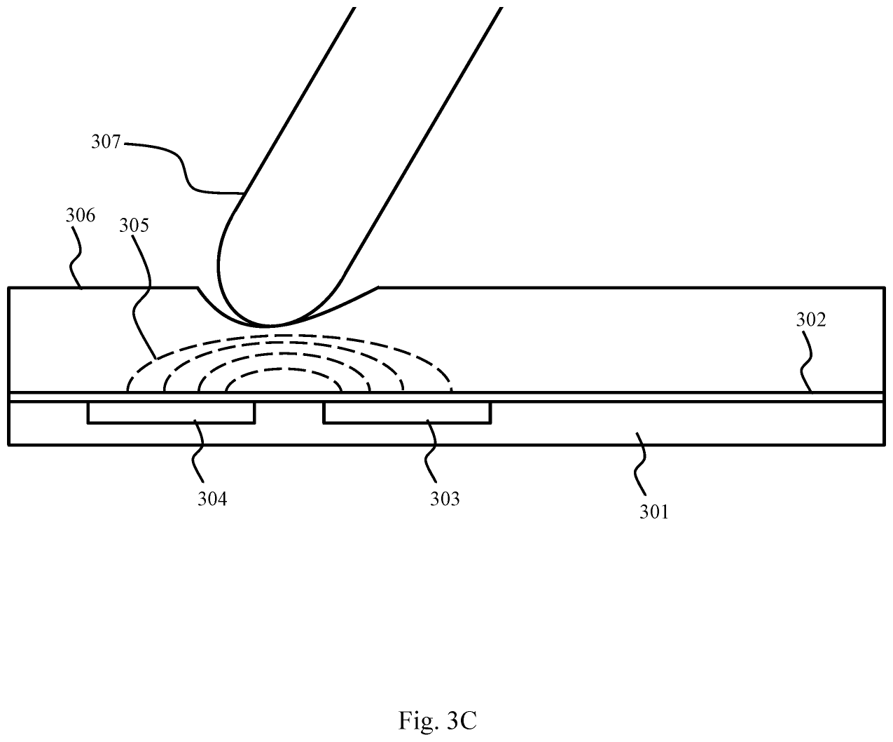

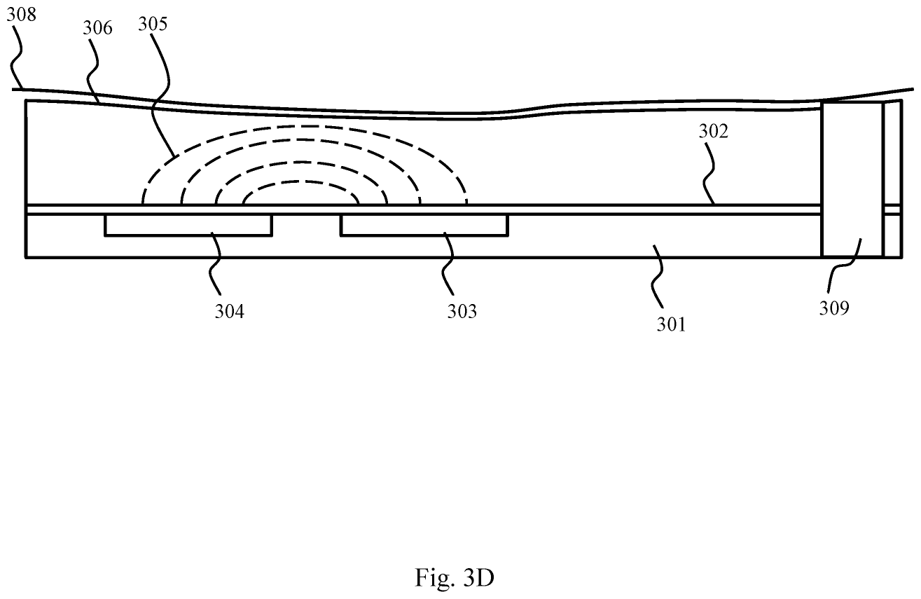

With reference now to FIG. 3A through FIG. 3D, an exemplary device of the present invention is shown in a cross-sectional view. In FIG. 3A, the base device includes a substrate 301 with a plurality of capacitive elements, including 303 and 304, as well as an insulating surface 302 disposed over the elements. Elements 303 and 304 are electrically isolated from one another, and when element 303 is held at a first potential and element 304 is held at a second potential, an electric field 305 is created between the two elements. In the example of FIG. 3A, the dielectric of the capacitor created between elements 303 and 304 is air. Insulating surface 302 may be made from any insulating material, including but not limited to an epoxy laminate material such as FR-4 or standard solder mask material. Insulating surface 302 may have a thickness of up to 80 microns.

With reference to FIG. 3B, an alternate design includes a membrane 306 placed over the insulating surface 302. The membrane 306 may be a homogenous mechanical and compressible, non-ferrite and non-conductive membrane, effectively replacing air as a dielectric for the capacitor created by two adjacent electrodes. The membrane may have a substantially uniform thickness, though in some embodiments the thickness may vary, for example a thickness may vary in order to make the device more form-fitting against a curved or irregular body region. The membrane provides a mechanical separation between the electrode surface and any external surface abutting the opposite surface of the membrane on a parallel plane to the surface facing the electrode plane. The membrane may be covered with a loose-fitting, non-conductive material, such as Nitrile.

The membrane 306 may be configured to have a substantially fixed and uniform density, compression ratio, and hardness across the entire substrate or exposed subset of the substrate. Suitable materials for use in a membrane of a device of the present invention include silicone, compressible foam, or saline encapsulators. The Shore hardness of the material may be in a range of Shore 00 0-20. The dielectric constant of the material may be in a range of 1.0 to approximately 5.0 The membrane material may be configured such that it has a steady state equilibrium shape that it returns to when not exposed to any external forces.

As shown in FIG. 3C, when an outside force is applied, for example with a finger 307, the membrane 306 will deform, changing the characteristics of electric field 305 and thus the capacitance of the capacitor created between capacitive elements 303 and 304. In the exemplary device, outside force is supplied by the normal pressure of the surface of the subject's skin or other tissue in the area being investigated. This change in capacitance can be measured using any suitable means in the art, for example with series or parallel resistive/inductive elements, or by measuring the impedance response of the capacitor itself or the capacitor in combination with other fixed or variable resistive, inductive, or capacitive elements. In this way, the location and degree of force exerted by the outside force may be measured and tracked over time.

Another example is shown in FIG. 3D, and shows an exemplary device being used in conjunction with an external surface 308, which has dielectric properties such that when placed on the membrane, the dielectric constant and hence the electric field pattern is disrupted between a pair of adjacent electrodes. This causes a change in the capacitance which can be measured for example using the methods outlined above. Suitable external surfaces for use with a device of the invention include, but are not limited to, human or other animal skin, human or other animal organs, or any ferrous or conductive or semi conductive material. When normal pressure from the surface of the tissue being investigated is applied between the external surface 308 and the capacitive elements 303 and 304, the membrane 306 deforms, as shown. When the pressure is not uniform, for example if the external surface 308 is a tissue having a variable firmness across the surface applied to the membrane 306, the membrane 306 will deform non-uniformly. The membrane may, for example, deform in a way that reflects underlying tissue structures, for example hard or soft lesions, A non-uniform membrane distortion across a surface having a plurality of capacitive elements will cause a measurable variation in the distance between the external surface and the electrode surface across the electrode array. The varying distances will affect the overall dielectric perceived by any adjacent pair capacitor, for example the adjacent pair 303 and 304. This will affect the dielectric constant of each capacitor, which can be used to calculate the distance between the external surface and the electrode surface across the array, which can in turn be used to calculate the formal force per square mm across the electrode surface. The change in capacitance is related to a change in perceived permittivity which is linearly proportional to the measured capacitance.

Also shown in FIG. 3D is electrically conductive element 309. Element 309 is configured to provide a stable reference potential to external surface 308. Element 309 may for example be referenced to a ground potential within the device, designed to provide a ground reference to the skin of the subject in the body region being evaluated. Alternatively, the ground reference may be obtained from conductive contact with the skin of the operator. A fixed reference potential on the external surface can in some embodiments greatly improve the quality of measurements across the electrode pairs. As contemplated herein, element 309 may be formed from any conductive material, including but not limited to copper, gold, silver, or platinum. In some embodiments, element 309 is made substantially from a first material, for example a metal, and coated on the end proximate to the external surface 308 with a second conductive or semiconductive material in order to provide better comfort for the user. In one embodiment, the membrane 306 defines a treatment area, for example a circular, ovular, square, rectangular or elliptical treatment area. The conductive element 309 may run substantially around the perimeter or edges of the treatment area, in order to maximize the chance of good electrical contact between the conductive element 309 and the external surface 308 (e.g. the skin of a subject).

Devices of the present invention may further include a controller comprising specialized circuitry for reading capacitance values from the various pairs of electrodes. The controller may comprise any computing device, for example an integrated microcontroller or processor, and may also comprise a quantity of volatile and/or non-volatile memory on which instructions may be stored to perform steps of a method of the present invention. In some embodiments, the controller comprises instructions configured to iterate through some or all possible electrode pairs provided in the array grid continuously, in order to provide a continuous measurement of the capacitance and therefore characteristics of the external surface 308 in contact with membrane 306. In some embodiments, the controller may utilize any of several commercially available chips or modules suitable for capacitive sensing and related calculations.

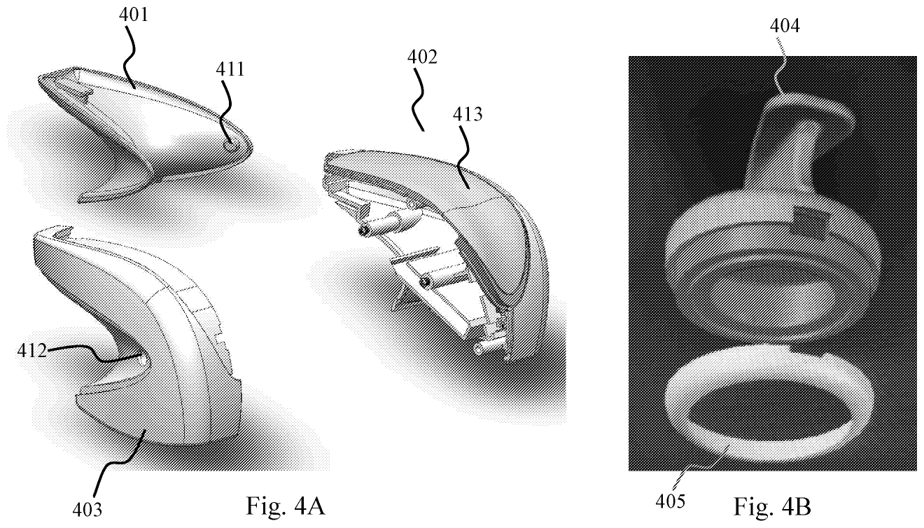

As described herein, embodiments of the system are configured to detect and evaluate subcutaneous soft tissue lesions by measuring and quantifying variations in tactile pressure at the surface of the tissue by means of capacitive sensing and measurements using a plurality of pairs (including at least one pair) of separate coplanar electrodes co-located on a substrate disposed within the housing, which are arranged in in a Cartesian grid, and configured such that any two adjacent electrodes are electrically independent and can be excited independently, creating a capacitor between the electrode pair. The system can therefore be used as a capacitive sensor. The sensor surface, in certain embodiments comprised of loose-fitting, non-conductive cover material, is placed over the outward facing surface of a compressible, non-ferrite and non-conductive membrane, positioned over the outward facing surface of the electrode grid, which serves as a dielectric for the capacitor created by any two adjacent electrodes (one functioning as the transmitting electrode and one functioning as the receiving electrode). The sensor surface is configured to be placed in contact with the tissue with sufficient pressure to slightly compress the compressible dielectric. The term “slightly compress” in certain instances can be the application of the minimal amount of pressure sufficient to ensure that the entire sensor surface is completely and firmly in contact with the tissue. At this point, the electromagnetic properties of the tissue will disrupt the electric field in the dielectric and disturb the capacitive coupling between the relevant pairs of coplanar electrodes. The disturbance in capacitance is measured utilizing a calibrated baseline set of measurements obtained by the operator from a lesion-free area of tissue, and by then determining the difference between such baseline measurements and the capacitive measurements obtained from each pair of electrodes, in the aggregate, facing the surface area of the tissue being interrogated. According to one embodiment, it is assumed that the electrical potential of the tissue in contact with the sensor, i.e. the patient, during a complete set of measurements (including baseline) remains constant, and not necessarily 0 Volts. It can also be assumed that the electrical potential of the tissue that is in contact with the device enclosure, i.e. the operator holding the device, is also constant during a complete set of measurements (including baseline), and again not necessarily 0 Volts. A classification of tissue stiffness is achieved by measuring differences in the tactile pressures of the surface of the tissue on the sensor surface. These pressures are then quantified by the measured capacitance between coplanar electrodes. The measured capacitance is compared to the baseline set of measurements, defined above and a relative measurement is then quantified for each set of electrodes. For the relative measurements to be consistent, it must be determined that the reference voltage potential used in both the baseline and the measured capacitance is exactly the same. This constant voltage potential can be obtained either (1) from the patient tissue or (2) the operator tissue as described above. Option (1) can be utilized by grounding options already described. Option (2) requires conductive surfaces to be placed on the housing of the device configured so that they will make contact with the operator at all times, including when making the baseline capacitive measurements and the measured capacitive measurements for relative tissue stiffness. With reference now to FIG. 4A and FIG. 4B, conductive surfaces are built onto the surface of portions of the housing (e.g. handheld portions) and are configured to provide the voltage reference from the operator. With reference to FIG. 4A, the exemplary housing includes a gripping surface 401 which connects to two housing portions 402 and 403. In the depicted embodiment, housing portion 402 includes a top gripping surface 413. In some embodiments, gripping surface 413 includes one or more buttons or other control elements allowing the operator to control the sensor assembly, for example to start or stop measurements or to zero out the sensor for calibration purposes. In the depicted embodiment, the gripping surface 401 is held in place by two pegs 411 (second peg is on the opposite internal surface of 401) mating with two corresponding holes 412 on the housing portions 402 and 403. In some embodiments, one or both of gripping surfaces 401 and 413 include a conductive element or finish which allows the sensor assembly to measure a reference voltage from the skin of the operator during use.

With reference to FIG. 4B, the exemplary housing includes a top handle surface 404 and a bottom ring 405. In various embodiments, either or both of handle 404 and ring 405 may include a conductive element or finish configured to measure a reference voltage from the skin of the operator (in the case of handle 404) or from the skin of the subject (ring 405). In some embodiments, the user and the subject are the same, e.g. during a self-examination.

Embodiments of the device may be incorporated into a housing, designed for self-testing. The integrated device may include a power source, for example a battery, power management hardware, and one or more communication devices, for example wired or wireless communication devices for transmitting or receiving data, configuration information, or operating instructions to and from a remote computing device. In one embodiment, the integrated device comprises a Bluetooth transceiver, and a remote computing device may be paired with the integrated device to send and receive data.

A system of the present invention comprises a sensor device as described above for detecting, documenting, measuring and mapping the size, shape and location of lesions underlying the surface of the skin or other soft tissue of a subject by measuring variations in the tactile pressure of the tissue surface in a body region of a subject, combined with a wired or wireless connection to a visualization computing device including software for visualizing the results, and in some embodiments for interpreting the results to provide a provisional analysis. The visualization computing device may include a wired or wireless transceiver for receiving, processing, displaying and storing data from the diagnostic device, which could be configured to use any wired or wireless communication protocol known in the art. In some embodiments, a data connection between the visualization device and the sensor device is encrypted.

In some embodiments, a system of the present invention is configured to perform a self-guided examination or evaluation by a subject. Such a system includes a guidance element, for example integrated into either or both of the sensor device and the visualization device described above. The guidance element may be configured to receive information from one or both of the sensor device and the visualization device and to deliver instructions or prompts to the subject based on that information. Instructions or prompts may include visual, auditory, or haptic feedback. In one example, a sensor device may be positioned on a tissue of a subject such that a structure of interest is detected along the top edge of the device. A guidance element may then issue an instruction to the subject to reposition the device, for example to move the device further up in order to better center the structure of interest in the detection area of the device. A guidance element may also instruct the user to relocate the device, for example, in order to span the entire breast. In some cases the guidance may instruct to switch from one breast to the other.

Another aspect of the invention includes a method of visualizing structures of interest in a tissue of a subject. Using a system as described above, a subject may position a sensor device having a detection surface on a body region, with the detection surface substantially in direct contact with the skin of the body region. Although not mandatory, the subject may then press a button or actuate some other control in order to begin the detection process. The device then gathers data from the tissue region in contact with the detection surface, and perform calculations based on that data. The processing may include denoising algorithms and basic smoothing algorithms. The processed data and/or the raw data is then transmitted via a wired or wireless connection to a visualization device, which may perform further calculations based on the data received. The finally processed data is then used to generate a visualization of the body region for viewing by the subject or by another, for example a clinician.

### Calibration

In one embodiment, systems and methods disclosed herein may include components and methods for performing dynamic, real-time baselining or calibration of array data obtained by an array of sensors, for example a cartesian array of capacitive and/or pressure sensors as disclosed herein. In one embodiment, a system includes a set of capacitive and/or pressure sensors positioned on a surface and arranged for example in a cartesian arrangement. A method may include obtaining capacitive and/or pressure data from the set of sensors periodically, for example at a rate of between 1 Hz and 1 kHz, or between 1 Hz and 500 Hz, or between 1 Hz and 100 Hz, or between 1 Hz and 30 Hz, or any suitable range. In one embodiment, the data obtained from the sensors is stored as a DC shifted sparse matrix, where a portion of the data is within 1.5 standard deviations from the mean. In one embodiment, most or the vast majority of the measured data is within 1.5 standard deviations of the mean.

A calibration method may include first collecting a quantity of data, then calculating a baseline calibration point or “zero” point from the data, wherein all data falling outside one standard deviation from the mean of the data is removed from consideration, and a second mean is calculated from the subset of data falling within one standard deviation (SD) of the first mean. Although the previous example uses one standard deviation from the mean as an exemplary threshold, it is understood that in other embodiments, any suitable threshold may be used, including but not limited to 0.3 SD, 0.5 SD, 0.75 SD, 0.8 SD, 0.9 SD, 1.1 SD, 1.25 SD, 1.5 SD, 2 SD, or the like. The second mean may then be used as a baseline or zero point for the remaining data. The second mean is then considered an offset for all the remaining data, and for each data point measured, the second mean is subtracted, resulting in the vast majority of the data being at or near the new zero point (i.e. the second mean), allowing the outlier data from the full set of measured data to be properly highlighted.

In some embodiments, a number of measurements are taken from all sensors before performing the initial calibration step, while in other embodiments the calibration is performed continuously, i.e. by performing the calibration on the very first set of data measured and repeating the calibration after each new set of data measured. This novel approach eliminates the need for the use of a conventional, single, static calibration, which can be inaccurate due to varying tissue stiffness between the tissue region used for calibration and the tissue in the measurement region. The method also prevents both inter-operator and intra-operator error in performing a single calibration incorrectly. For example, in some embodiments an operator may apply varying pressure to the tissue between measurements or over the course of a single measurement, which would result in skewed data if a static calibration was used. By dynamically calibrating the data as it is measured, the overall bias applied to the data can be adapted to properly differentiate true outliers from skew caused by varying operator pressure.

In one embodiment, the calculated baseline level is used to validate and store a quantity of the measured data. In one example, a quantity of data points is measured from at least one pressure or capacitive sensor arranged in an array, for example a Cartesian array. In one embodiment, a moving average of the measured data points is calculated in order to smooth the data over time. The moving average may be calculated in a window of at least three samples, at least five samples, at least ten samples, at least 20 samples, or any suitable number. The smoothed moving average data may then be monitored, and when the moving average moves into a certain range, a capture trigger may be activated to indicate that the captured data is now valid for measurement. In some embodiments, the certain range may be between 40% and 60% of the dynamic measurement range of the sensors, or between 45% and 55%, between 47% and 53%, between 49% and 51%, or about 50% of the dynamic measurement range of the sensor. In some embodiments, once the capture trigger is activated, the data is then monitored to see whether it stays valid for a period of time, for example two seconds, three seconds, or five seconds. When the data stays valid for the specified period of time, the smoothed and/or raw data measured during that interval is then stored as a valid data measurement.

### Sample Data Visualization

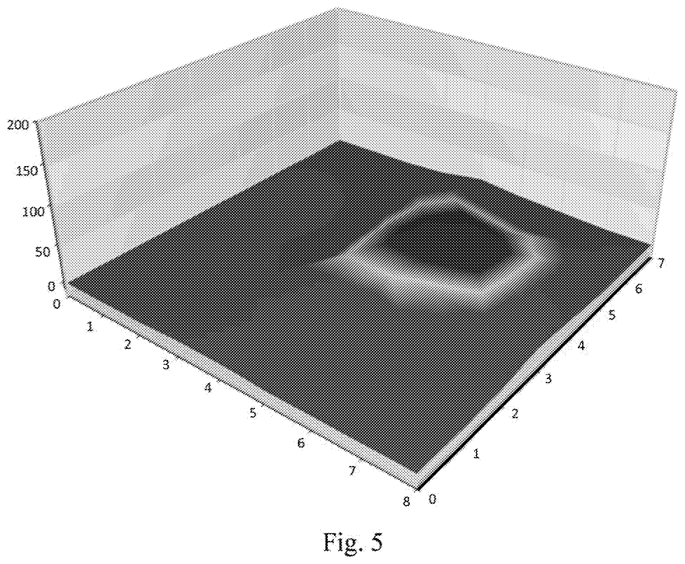

In some embodiments and with reference now to FIG. 5, in addition to the visualization of the body region, the visualization device may display some indication of preliminary or actual screening determinations. In some embodiments, the diagnostic device performs all data gathering and processing, and the results are sent via a wired or wireless connection to a remote computing system or cloud system for processing and later visualization.

In some embodiments, a method of the present invention may include one or more guided steps, for example indications or prompts delivered by the visualization device or the sensor device to the subject or a clinician, including steps to guide the subject through a self-screening. Such methods may include the step of processing some or all of the data measured by the sensor device, either by a processor integrated into the device, by a processor integrated into the visualization device, or by another device located near or remote from the diagnostic system comprising the diagnostic device and the visualization device. The guided steps may further include indicating to the subject or clinician, based on the processed data, one or more manipulation steps for the subject or clinician to perform on or with the device, for example move up, move down, move left, move right, press harder, press more lightly With such a self-screening system, it is possible for a subject having no or minimal training in medicine or with the device to perform a guided self-screening in order to gather data to be reviewed by a clinician.

In some embodiments, the graphic visualization may include or be updated based on the dynamic calibration methods disclosed above. In some embodiments, a 3D surface plot may be shown by an interface of the invention, where the surface of the tissue being measured is displayed in the X-Y plane, and the data is represented on the Z axis. As the baseline is updated, the X-Y surface of the data plot is vertically offset along the Z axis by subtracting the calculated baseline quantity. Color coding of the surface is used to indicate the deviation from an optimal baseline. When the baseline is in the optimal range, the surface appears green, except for aberrations which appear as yellow and red progressively. As the baseline deviates from an optimal value, the surface transitions to yellow and eventually to red, indicating substantial deviation from an optimal baseline. In addition, in some embodiments, a calibration display may show the calculated baseline itself graphically on the screen, providing further feedback to the operator allowing the operator to (for example) adjust their applied pressure to move the baseline into the optimal range. This novel visualization approach optimizes user training by facilitating the capture of consistent baselines and maximizing the integrity of the measurements.

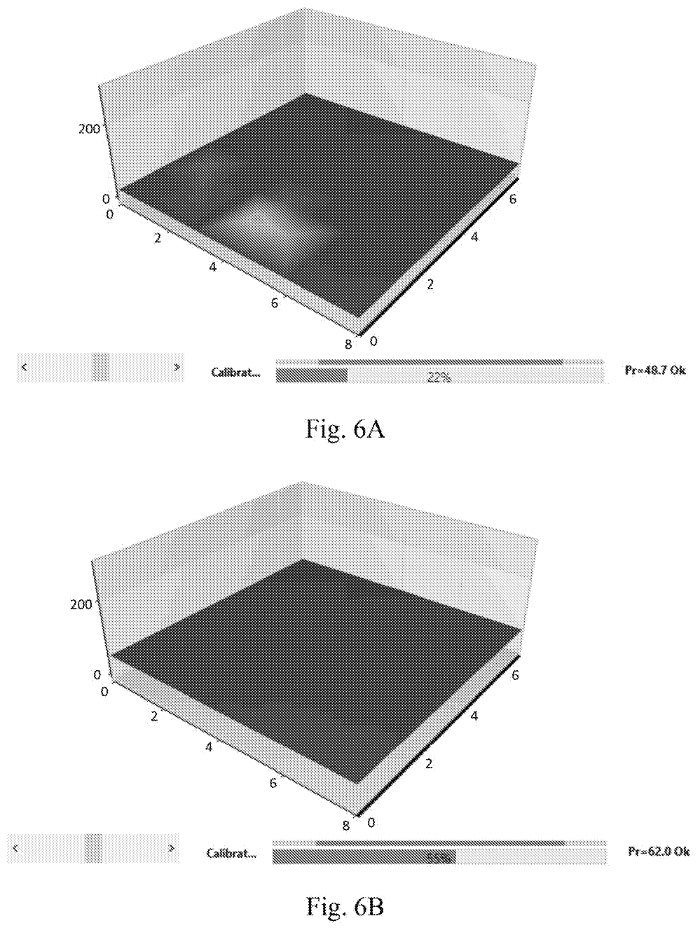

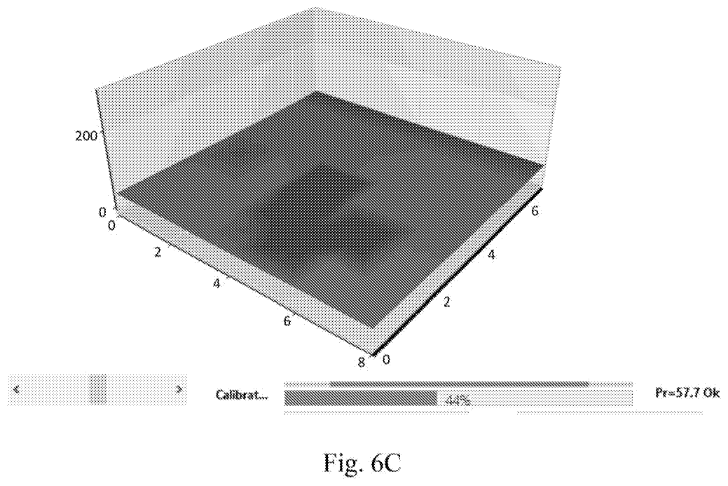

One example is shown with reference to FIG. 6A, FIG. 6B, and FIG. 6C. As shown in FIG. 6A, an uncalibrated surface map may show most points in red, i.e. aberrations from a fixed “zero” value. In the depicted example, most points in the surface map are measured around 22%, which is significantly below the fixed zero value of 50%. The points therefore are all highlighted in red as aberrations from the zero value.

FIG. 6B shows a visualization of dynamically calibrated data. The data map shown in FIG. 6B shows the entire surface in green, i.e. normal. The data has been calibrated to remove offsets, and so most falls within the green window, which in the depicted embodiment is between 55% to 65%.

FIG. 6C shows a visualization of data approaching optimal real-time calibration. Much of the graph is shown in green, but some of the surface is yellow-green, indicating that some of the data still falls outside the zero window. The mean of the depicted data is about 44%, which falls just outside the optimal window of 45% to 55%.

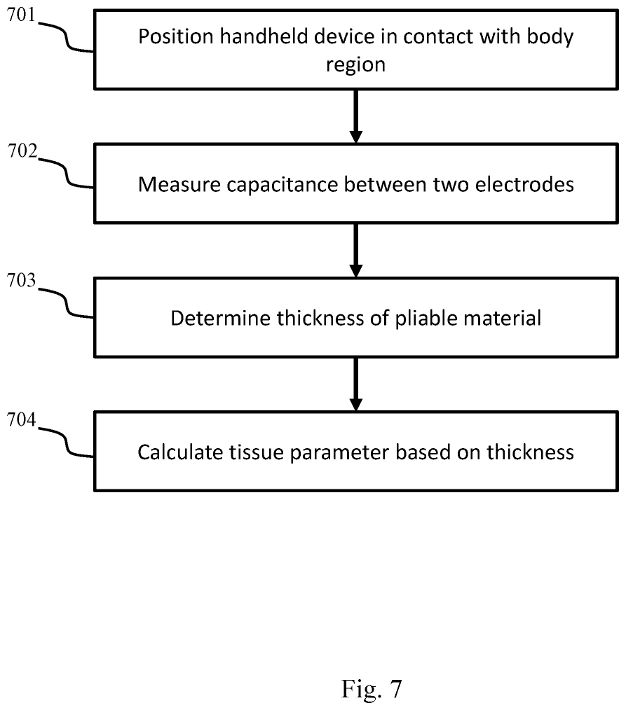

An exemplary method of measuring a tissue parameter in a subject is shown in FIG. 7. The method includes the steps of positioning a handheld device comprising a plurality of electrodes and a quantity of non-conductive compressible material and covering substantially in contact with a body region of a subject in step 701, measuring a capacitance between two electrodes of the plurality of electrodes in step 702, determining, based on the capacitance, the thickness of the pliable material in step 703, and calculating a tissue parameter based on the thickness of the pliable material in step 704.

According to one embodiment, a capacitive tactile sensor device for detecting, documenting, measuring and mapping the size, shape and location of lesions underlying the surface of the skin or other soft tissue in a subject by measuring variations in the tactile pressure of the skin or other tissue surface, includes a housing having at least one exposure window and exposed conductive surfaces placed so that the operator or the device will be in contact with the conductive surfaces at all times while operating the device. A substrate is located within the housing measurements comprising a plurality of pairs of separate coplanar, co-located electrodes, arranged in in a Cartesian grid, on an electrically non-conductive material, and configured such that any two adjacent electrodes are electrically independent and can be stimulated independently by an externally generated stimulating voltage or current signal, creating a capacitor between each such electrode pair, with the space above the outward surface of the electrode grid serving as a dielectric. A layer of insulating material is positioned over the outward facing surface of the electrode grid. A homogeneous, compressible, non-ferrite and non-conductive membrane covering the insulating layer is positioned over the outward facing surface of the electrode grid, which membrane: (a) is of uniform thickness across the entire surface area of each pair of adjacent electrodes forming a capacitor, and across the entire surface area of the Cartesian grid of electrodes; (b) has a fixed, uniform and known compression ratio and hardness across the entire surface area of the Cartesian grid of electrodes; and (c) has a steady-state equilibrium shape such that, when not subject to external forces, its volume remains constant. A non-conductive cover material is positioned over the outward facing surface of the homogeneous, compressible, non-ferrite and compressible membrane, at least a portion of which is accessible through the exposure window of the housing, which serves as the outward facing surface of the sensor and which, together with the membrane, effectively replaces air as a dielectric for the capacitor created by each pair of adjacent electrodes, one functioning as the transmitting electrode and one functioning as the receiving electrode. The sensor surface is configured to be placed completely and firmly in contact with the surface of the tissue of the subject such that the electromechanical properties including normal surface pressure of the conductive tissue will disrupt the electric field in the dielectric comprised of the compressible membrane and disturb the capacitive coupling between the relevant pairs of coplanar electrodes; wherein the sensor input device induces and generates at least one signal in response to the pressure imposed upon the sensor surface by contact with the skin or other soft tissue surface, and the resulting compression of the compressible membrane and disturbance of the capacitive coupling between the relevant pairs of coplanar electrodes, in accordance with the varying composition and properties of the underlying tissue structure, as an indicator of the location, dynamic and special features of localized areas of stiffer tissue underlying the area of skin or other tissue surface being investigated. A controller is disposed within the housing comprised of: (a) one or more commercially available IC capacitive sensor chips configured: (i) to generate signals used to stimulate the electrode pairs located in the substrate; (ii) to receive and process the signals subsequently induced, generated and transmitted by the capacitive tactile sensor input device in response to the pressure imposed upon the sensor surface by contact with the skin or other soft tissue surface; and (iii) to induce, generate and transmit to the device signals derived from the processing of the signals induced, generated and transmitted to the IC capacitive sensor chip(s) by the capacitive tactile sensor input device; and (b) one or more integrated circuits and processors configured (i) to receive and process the signals induced, generated and transmitted by the IC capacitive sensor chip(s) to calculate one or more parameters of the underlying tissue structure, based on changes in the perceived capacitance between the adjacent relevant electron pairs caused by the pressure imposed upon the sensor surface by contact with the skin or other soft tissue surface; and (ii) to communicatively connect with a visualization device comprising a display and an integrated storage device.

In one embodiment, the homogeneous, compressible, non-ferrite and non-conductive membrane comprises a polyurethane, silicon or thermoplastic elastomer foam. In one embodiment, the foam has a hardness in a range of 00-0 to 00-20. In one embodiment, the plurality of electrodes comprises at least 2 electrodes. In one embodiment, each of the plurality of electrodes has a surface area of between 1 mm² and 16 mm². In one embodiment, the device includes a visualization device communicatively connected to the controller, the visualization device comprising a display. In one embodiment, the controller is connected to the visualization device via a Bluetooth connection. In one embodiment, the device includes conducting elements on the exterior of the housing configured to deliver a voltage reference used for all subsequent capacitive calculations. In one embodiment, the device includes a grounding pad or strap providing conductive contact between an uninsulated portion of the electron plane and the external surface of the capacitive sensor, configured to deliver a voltage reference used for all subsequent capacitive calculations. In one embodiment, the at least one parameter comprises tissue firmness. In one embodiment, the tissue of the subject comprises breast tissue.

In one embodiment, a method of documenting, measuring and mapping the size, shape and location of palpable lesions underlying the surface of the skin or other soft tissue in a subject by measuring variations in the normal tactile pressure on the surface of the skin or other soft tissue areas of the tissue surface using capacitive tactile sensing, includes the steps of positioning a handheld device comprising a plurality of electrodes and a quantity of non-conductive compressible material and covering substantially in contact with the surface area of the skin or tissue of a subject; determining a baseline reference capacitance of the subject by measurements obtained by the operator from a lesion-free area of tissue; determining a baseline electrical voltage potential reference from the operator through conductive contact between the operator and the handheld device; positioning the handheld device substantially in contact with the surface area of the skin or tissue being investigated; measuring capacitance between two electrodes of the plurality of electrodes resulting from contact with the surface area of the skin or tissue being investigated; measuring variations in the perceived capacitance between the adjacent relevant electrode pairs, with reference to the baseline electrical voltage potential references, caused by the pressure imposed upon the sensor surface (compressible membrane and covering), resulting from the disruption to the capacitive coupling between the adjacent relevant electrode pairs, caused by contact with the skin or surface of the tissue being investigated; and calculating one or more tissue parameters based on the variations in perceived capacitance from the baseline electrical voltage potential references. In one embodiment, the method includes the step of transmitting data selected from the group consisting of the perceived capacitance, the thickness, and the tissue parameter to a visualization system. In one embodiment, the method includes the step of measuring a plurality of capacitance values between a set of electrode pairs, and displaying on the visualization system a diagram of parameter values across the tissue surface. In one embodiment, the tissue parameter is firmness.

In one embodiment, a method of performing a guided self-examination on a tissue of a subject includes the steps of positioning a handheld device comprising a plurality of electrodes and a quantity of pliable material substantially in contact with a body region of a subject; measuring a capacitance between two electrodes of the plurality of electrodes; calculating a tissue parameter based on the capacitance; determining, in a controller, an instruction for the subject based on the tissue parameter; and issuing the instruction to the subject in order to induce the subject to manipulate the handheld device. In one embodiment, the instruction is an auditory instruction issued via a speaker. In one embodiment, the instruction is configured to induce the subject to move the device to a new location on the tissue. In one embodiment, the instruction comprises a visual cue, presented on a display.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

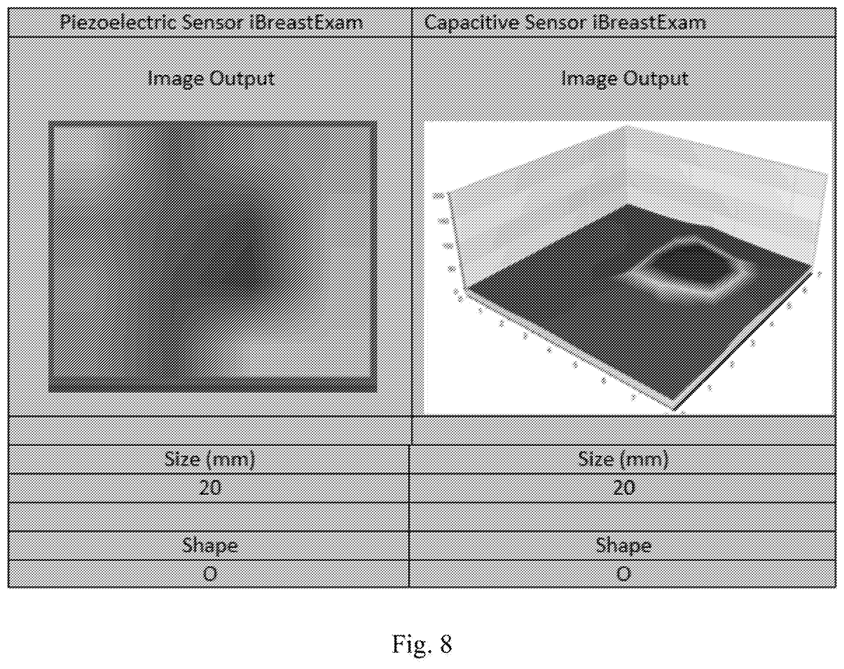

A sample comparison data for piezo vs. capacitive sensor is provided with reference to FIG. 8 according to one embodiment.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A device for measuring a tissue parameter in a subject, comprising:
a housing having at least one exposure window;
a substrate comprising a plurality of electrodes disposed within the housing;
an insulating layer positioned over the electrodes;
a pliable membrane positioned over the insulating layer wherein at least a portion of the pliable membrane is accessible via the exposure window; and
a controller disposed within the housing configured to calculate at least one parameter of a tissue of a subject based on a capacitance measured between the electrodes.

2. The device of claim 1, wherein the pliable membrane comprises a foam.

3. The device of claim 2, wherein the foam has a hardness in a range of 00-0 to 00-20.

4. The device of claim 1, wherein the plurality of electrodes comprises at least 2 electrodes.

5. The device of claim 1, wherein each of the plurality of electrodes has a surface area of between 1 mm² and 16 mm².

6. The device of claim 1, further comprising a visualization device communicatively connected to the controller, the visualization device comprising a display.

7. The device of claim 6, wherein the controller is connected to the visualization device via a Bluetooth connection.

8. The device of claim 1, further comprising a conducting element configured to deliver a voltage reference to a surface in contact with the pliable membrane.

9. The device of claim 8, wherein the conducting element is positioned substantially surrounding the exposure window.

10. The device of claim 1, wherein the at least one parameter comprises tissue firmness.

11. The device of claim 1, wherein the tissue of the subject comprises breast tissue.

12. A method of measuring a tissue parameter in a subject, comprising:
positioning a handheld device comprising a plurality of electrodes and a quantity of pliable material substantially in contact with a body region of a subject;
measuring a capacitance between two electrodes of the plurality of electrodes;
determining, based on the capacitance, the thickness of the pliable material; and
calculating a tissue parameter based on the thickness of the pliable material.

13. The method of claim 12, further comprising transmitting data selected from the group consisting of the capacitance, the thickness, and the tissue parameter to a visualization system.

14. The method of claim 13, further comprising measuring a plurality of capacitance values between a set of electrode pairs, and displaying on the visualization system a diagram of parameter values across the tissue surface.

15. The method of claim 12, wherein the tissue parameter is firmness.

16. The method of claim 12, further comprising the step of making a provisional diagnosis based on the tissue parameter.

17. A method of performing a guided self-examination on a tissue of a subject, comprising:
positioning a handheld device comprising a plurality of electrodes and a quantity of pliable material substantially in contact with a body region of a subject;
measuring a capacitance between two electrodes of the plurality of electrodes;
calculating a tissue parameter based on the capacitance;
determining, in a controller, an instruction for the subject based on the tissue parameter; and
issuing the instruction to the subject in order to induce the subject to manipulate the handheld device.

18. The method of claim 17, wherein the instruction is an auditory instruction issued via a speaker.

19. The method of claim 17, wherein the instruction is configured to induce the subject to move the device to a new location on the tissue.

20. The method of claim 17, wherein the instruction comprises a visual cue, presented on a display.

21. A capacitive tactile sensor device for detecting, documenting, measuring and mapping the size, shape and location of lesions underlying the surface of the skin or other soft tissue in a subject by measuring variations in the tactile pressure of the skin or other tissue surface, comprising:
a housing having at least one exposure window and exposed conductive surfaces placed so that the operator or the device will be in contact with the conductive surfaces at all times while operating the device;
a substrate located within the housing measurements comprising a plurality of pairs of separate coplanar, co-located electrodes, arranged in in a Cartesian grid, on an electrically non-conductive material, and configured such that any two adjacent electrodes are electrically independent and can be stimulated independently by an externally generated stimulating voltage or current signal, creating a capacitor between each such electrode pair, with the space above the outward surface of the electrode grid serving as a dielectric;
a layer of insulating material positioned over the outward facing surface of the electrode grid;
a homogeneous, compressible, non-ferrite and non-conductive membrane covering the insulating layer positioned over the outward facing surface of the electrode grid, which membrane: (a) is of uniform thickness across the entire surface area of each pair of adjacent electrodes forming a capacitor, and across the entire surface area of the Cartesian grid of electrodes; (b) has a fixed, uniform and known compression ratio and hardness across the entire surface area of the Cartesian grid of electrodes; and (c) has a steady-state equilibrium shape such that, when not subject to external forces, its volume remains constant; and
a non-conductive cover material positioned over the outward facing surface of the homogeneous, compressible, non-ferrite and compressible membrane, at least a portion of which is accessible through the exposure window of the housing, which serves as the outward facing surface of the sensor and which, together with the membrane, effectively replaces air as a dielectric for the capacitor created by each pair of adjacent electrodes, one functioning as the transmitting electrode and one functioning as the receiving electrode;
wherein the sensor surface is configured to be placed completely and firmly in contact with the surface of the tissue of the subject such that the electromechanical properties including normal surface pressure of the conductive tissue will disrupt the electric field in the dielectric comprised of the compressible membrane and disturb the capacitive coupling between the relevant pairs of coplanar electrodes;
wherein the sensor input device induces and generates at least one signal in response to the pressure imposed upon the sensor surface by contact with the skin or other soft tissue surface, and the resulting compression of the compressible membrane and disturbance of the capacitive coupling between the relevant pairs of coplanar electrodes, in accordance with the varying composition and properties of the underlying tissue structure, as an indicator of the location, dynamic and special features of localized areas of stiffer tissue underlying the area of skin or other tissue surface being investigated; and
a controller disposed within the housing comprised of: (a) one or more commercially available IC capacitive sensor chips configured: (i) to generate signals used to stimulate the electrode pairs located in the substrate; (ii) to receive and process the signals subsequently induced, generated and transmitted by the capacitive tactile sensor input device in response to the pressure imposed upon the sensor surface by contact with the skin or other soft tissue surface; and (iii) to induce, generate and transmit to the device signals derived from the processing of the signals induced, generated and transmitted to the IC capacitive sensor chip(s) by the capacitive tactile sensor input device; and (b) one or more integrated circuits and processors configured (i) to receive and process the signals induced, generated and transmitted by the IC capacitive sensor chip(s) to calculate one or more parameters of the underlying tissue structure, based on changes in the perceived capacitance between the adjacent relevant electron pairs caused by the pressure imposed upon the sensor surface by contact with the skin or other soft tissue surface; and (ii) to communicatively connect with a visualization device comprising a display and an integrated storage device.

22. The device of claim 21, wherein the homogeneous, compressible, non-ferrite and non-conductive membrane comprises a polyurethane, silicon, or thermoplastic elastomer foam.

23. The device of claim 22, wherein the foam has a hardness in a range of 00-0 to 00-20.

24. The device of claim 21, wherein the plurality of electrodes comprises at least 2 electrodes;

25. The device of claim 21, wherein each of the plurality of electrodes has a surface area of between 1 mm² and 16 mm².

26. The device of claim 21, further comprising a visualization device communicatively connected to the controller, the visualization device comprising a display.

27. The device of claim 26, wherein the controller is connected to the visualization device via a Bluetooth connection.

28. The device of claim 21, further comprising conducting elements on the exterior of the housing configured to deliver a voltage reference used for all subsequent capacitive calculations.

29. The device of claim 21, including a grounding pad or strap providing conductive contact between an uninsulated portion of the electron plane and the external surface of the capacitive sensor, configured to deliver a voltage reference used for all subsequent capacitive calculations.

30. The device of claim 21, wherein the at least one parameter comprises tissue firmness.

31. The device of claim 21, wherein the tissue of the subject comprises breast tissue.

32. A method of documenting, measuring and mapping the size, shape and location of palpable lesions underlying the surface of the skin or other soft tissue in a subject by measuring variations in the normal tactile pressure on the surface of the skin or other soft tissue areas of the tissue surface using capacitive tactile sensing, comprising:
positioning a handheld device comprising a plurality of electrodes and a quantity of non-conductive compressible material and covering substantially in contact with the surface area of the skin or tissue of a subject;
determining a baseline reference capacitance of the subject by measurements obtained by the operator from a lesion-free area of tissue;
determining a baseline electrical voltage potential reference from the operator through conductive contact between the operator and the handheld device;
positioning the handheld device substantially in contact with the surface area of the skin or tissue being investigated;
measuring capacitance between two electrodes of the plurality of electrodes resulting from contact with the surface area of the skin or tissue being investigated;
measuring variations in the perceived capacitance between the adjacent relevant electrode pairs, with reference to the baseline electrical voltage potential references, caused by the pressure imposed upon the sensor surface (compressible membrane and covering), resulting from the disruption to the capacitive coupling between the adjacent relevant electrode pairs, caused by contact with the skin or surface of the tissue being investigated; and
calculating one or more tissue parameters based on the variations in perceived capacitance from the baseline electrical voltage potential references.

33. The method of claim 32, further comprising transmitting data selected from the group consisting of the perceived capacitance, the thickness, and the tissue parameter to a visualization system.

34. The method of claim 33, further comprising measuring a plurality of capacitance values between a set of electrode pairs, and displaying on the visualization system a diagram of parameter values across the tissue surface.

35. The method of claim 32, wherein the tissue parameter is firmness.

36. The method of claim 32 utilizing the device in claim 31.

37. A method of performing a guided self-examination on a tissue of a subject, comprising:
positioning a handheld device comprising a plurality of electrodes and a quantity of pliable material substantially in contact with a body region of a subject;
measuring a capacitance between two electrodes of the plurality of electrodes;
calculating a tissue parameter based on the capacitance;
determining, in a controller, an instruction for the subject based on the tissue parameter; and
issuing the instruction to the subject in order to induce the subject to manipulate the handheld device.

38. The method of claim 37, wherein the instruction is an auditory instruction issued via a speaker.

39. The method of claim 37, wherein the instruction is configured to induce the subject to move the device to a new location on the tissue.

40. The method of claim 37, wherein the instruction comprises a visual cue, presented on a display.

41. A method of dynamically calibrating data received from at least one sensor, comprising:
acquiring a plurality of data values from at least one sensor;
calculating a first mean and a first standard deviation of the plurality of data values;
obtaining a subset of the plurality of data values, whose values are within one standard deviation of the first mean;
calculating a second mean from the subset of the plurality of data values as a baseline value; and
subtracting the baseline value from the plurality of data values to generate a calibrated data set.

42. The method of claim 41, further comprising:
acquiring a second plurality of data values;
calculating a third mean and third standard deviation of the first and second pluralities of data values;
obtaining a second subset of the first and second pluralities of data values, whose values are within one standard deviation of the third mean;
calculating a fourth mean from the second subset of the pluralities of data values as a second baseline value; and
subtracting the second baseline value from the first and second pluralities of data values to generate the calibrated data set.

43. The method of claim 41, further comprising:
displaying a three-dimensional surface plot of the calibrated data set;
displaying baseline value; and
providing visual feedback when the baseline value is within an optimal range.

44. The method of claim 43, wherein the visual feedback comprises changing a color of a region of the three-dimensional surface plot.

45. The method of claim 41, further comprising calculating a moving average of the plurality of data values and calculating the first mean and first standard deviation from the moving average.

46. The method of claim 45, further comprising:
comparing the moving average to a maximum and a minimum threshold;
capturing the plurality of data values or the moving average when the moving average is between the maximum and minimum thresholds; and
storing the captured data values on a non-transitory computer-readable medium when the moving average remains between the maximum and minimum thresholds for a specified period of time.

47. The method of claim 46, wherein the specified period of time is at least three seconds.
